(19) United States
(12) Patent Application Publication
(10) Pub. No.: US 2010/0029017 A1
(43) Pub. Date: Feb. 4, 2010

(54) Mono-chlorinated hydroxycoumarin conjugates

(75) Inventors: Zhenjun Diwu, Sunnyvale, CA (US); Timothy Dubrovsky, Davis, CA (US); Barnaby Abrams, San Carlos, CA (US); Jianjun He, Sunnyvale, CA (US); Chunmei Wei, Cupertino, CA (US); Jinfang Liao, Foster City, CA (US)

Correspondence Address: David W. Highet, VP & Chief IP Counsel;Becton, Dickinson and Company, 1 Becton Drive, MC 110, Franklin Lakes, NJ, 07417-1880, US

(73) Assignee: Becton, Dickinson and Company

(21) Appl. No.: 12/220,939

(22) Filed: Jul. 29, 2008

(51) Int. Cl. G01N 33/536; C07K 16/18

(52) U.S. Cl. 436/536; 530/391.3

(57) ABSTRACT

The present invention provides fluorescent biopolymers that are amino acid polymers conjugated to multiple mono-chlorinated 3-carbonyl-7-hydroxy-coumarin dyes, and methods of their use. The fluorescent biopolymers containing multiple mono-chlorinated 3-carbonyl-7-hydroxy-coumarin dyes generally exhibit superior fluorescence properties compared to biopolymers labeled with multiple non-chlorinated hydroxycoumarin dyes. The fluorescent biopolymers of the invention in which the amino acid polymer is an antigen-specific antibody are particularly useful for analyzing cells by flow cytometers that are equipped with a violet laser as an excitation source due to their strong absorption at 405 nm and high fluorescence quantum yield.

### BACKGROUND OF THE INVENTION

1. Field of the Invention

The invention relates to fluorescently labeled biopolymers and their use, preferably as detection reagents.

2. Description of the Related Art

Analyte-specific fluorescent probes are useful reagents for the analysis of analytes in a sample. The analytes become labeled through specific binding to the probes, and the labeling facilitates detection of the analyte. Applications of fluorescent probes for the analysis of analytes in a sample include fluorescence immunoassays, including the identification and/or separation of subpopulations of cells in a mixture of cells by flow cytometry, fluorescence microscopy, and visualization of gel separated analytes by fluorescence staining. These techniques are described by Herzenberg et al., “CELLULAR IMMUNOLOGY” 3rd ed., Chapter 22; Blackwell Scientific Publications (1978); and by Goldman, “FLUORESCENCE ANTIBODY METHODS” Academic Press, New York, (1968); and by Taylor et al., APPLICATIONS OF FLUORESCENCE IN THE BIOMEDICAL SCIENCES, Alan Liss Inc., (1986), each of which is incorporated herein by reference.

When employing fluorescent dyes for the above purposes, there are many constraints on the choice of the fluorescent dye. One constraint is the absorption and emission characteristics of the fluorescent dye, since many ligands, receptors, and materials in the sample under test, e.g., blood, urine, cerebrospinal fluid, will fluoresce and interfere with an accurate determination of the fluorescence of the fluorescent label. This phenomenon is called autofluorescence or background fluorescence. Another consideration is the ability to conjugate the fluorescent dye to ligands and receptors and other biological and non-biological materials and the effect of such conjugation on the fluorescent dye. In many situations, conjugation to another molecule may result in a substantial change in the fluorescent characteristics of the fluorescent dye and, in some cases, substantially destroy or reduce the quantum efficiency of the fluorescent dye. It is also possible that conjugation with the fluorescent dye will inactivate the function of the molecule that is labeled. A third lo consideration is the quantum efficiency of the fluorescent dye which should be high for sensitive detection. A fourth consideration is the light absorbing capability, or extinction coefficient, of the fluorescent dyes, which should also be as large as possible. Also of concern is whether the fluorescent molecules will interact with each other when in close proximity, resulting in self-quenching. An additional concern is whether there is non-specific binding of the fluorescent dye to other compounds or container walls, either by themselves or in conjunction with the compound to which the fluorescent dye is conjugated.

The applicability and value of the methods indicated above are closely tied to the availability of suitable fluorescent compounds. In particular, there is a need for fluorescent substances that can be excited by the commercial viable laser sources such as the violet laser (405 nm), argon laser (488 nm) and He—Ne laser (633 nm). There are many fluorescent dyes developed for argon laser (488 nm excitation) and He—Ne laser (633 nm excitation). For example, fluorescein, which is well excited by 488 nm argon laser, is a useful emitter in the green region. However, there are few fluorescent dyes available for the 405 nm violet laser.

Phycobiliproteins have made an important contribution because of their high extinction coefficient and high quantum yield. These chromophore-containing proteins can be covalently linked to many proteins and are used in fluorescence antibody assays in microscopy and flow cytometry. The phycobiliproteins have the disadvantages that (1) the protein labeling procedure is relatively complex; (2) the protein labeling efficiency is not usually high (typicaily an average of 0.5 phycobiliprotein molecules per protein); (3) the phycobiliprotein is a natural product and its preparation and purification is complex; (4) the phycobiliproteins are expensive; (5) there are at present no phycobiliproteins available as labeling reagents that are excited at 405 run violet laser; (6) the phycobiliproteins are large proteins with molecular weights ranging from 33,000 to 240,000 and are larger than many materials that it is desirable to label, such as metabolites, drugs, hormones, derivatized nucleotides, and many proteins including antibodies. The latter disadvantage is of particular importance because antibodies, avidin, DNA-hybridization probes, hormones, and small molecules labeled with the large phycobiliproteins may not be able to bind to their targets because of steric limitations imposed by the size of the conjugated complex.

Fluorescent compounds are covalently or noncovalently attached to other materials to impart color and fluorescence. Brightly fluorescent dyes permit detection or location of the attached materials with great sensitivity. Certain coumarin dyes have demonstrated utilities for a variety of biological detection applications, e.g., U.S. Pat. No. 6,207,404 to Miller, et al. (2001); U.S. Pat. No. 5,830,912 to Gee et al. and U.S. Pat. No. 4,956,480 to Robinson (1990). In particular, the much smaller size of coumarins (compared to other fluorescent labeling dyes such as fluoresceins, rhodamines and cyanines) minimize the effect of external tags on the affinity and specificity of ligands (to their receptors). In addition, the smaller coumarins have higher labeling efficiency than fluoresceins, rhodamines and cyanines. Nevertheless, many coumarin dyes are known to share certain disadvantages, e.g., severe quenching of the fluorescence of hydroxyl coumarin dyes on protein conjugates due to their strong hydrophobicity and high pKa. The coumarin fluorescence quenching is resulted from self-quenching (close distance between coumarin tags) and/or from the quenching by electron-rich amino acid residues (such as histidine, tryptophan and tyrosine etc). In addition, the weak absorption at 405 nm of coumarins severely limits their applications for analyzing cells by violet laser excitation such as flow cytometers.

Chlorinated coumarins have been used to label small organic molecules (e.g., Zlokarnik et al., 1998, Science 279:84 and U.S. Pat. No. 5,955,604). However, it has not been reported that chlorinated coumarins could be used for labeling biopolymers, such as antibodies. For labeling small organic molecules, neither self-quenching nor the quenching by the substrate is a severe problem because only a single coumarin molecule is present in each conjugate. In contrast, for labeling antibodies, both self-quenching and quenching by the substrate are a significant concern because it is desirable to conjugate multiple dye molecules to each antibody. It is generally considered that chlorination of coumarins or fluoresceins would result in inferior labeling of antibodies due to the so called “heavy atom effect” and increased hydrophobicity (U.S. Pat. No. 5,516,629 to Park et al.; U.S. Pat. No. 5,830,912 to Gee et al.; U.S. Pat. No. 6,472,205 to Tsien and Zlokarnik; Haugland, Handbook of Fluorescent Probes and Research Chemicals, Molecular Probes, 9^{th} ed., pp 7-74, 2002).

### SUMMARY OF THE INVENTION

The present invention provides fluorescent biopolymers that are biopolymers conjugated to a plurality of mono-chlorinated, 7-hydroxycoumarin dyes. The fluorescent biopolymers are derived from reactive 3-carbonyl-6-chloro-7-hydroxycoumarins dyes or 3-carbonyl-8-chloro 7-hydroxycoumarins dyes.

The fluorescent biopolymers of the present invention exhibit combinations of properties that were unexpected in view of prior teachings in the field, as exemplified by the references cited above, and that make these fluorescent biopolymers particularly suited for use in biological assays. The mono-chlorinated hydroxycoumarin dye unexpectedly results in decreased self-quenching of the fluorescence of biopolymers conjugated to a multiplicity of the dye. The mono-chlorinated hydroxycoumarin dye exhibits a significantly decreased pKa, such that the fluorescent biopolymers of the present invention exhibit maximum fluorescence in the range of physiological pH. Under a range of conditions useful for typical biological assays, biopolymers conjugated to multiple mono-chlorinated hydroxycoumarins are substantially more fluorescent than comparable biopolymers conjugated to multiple structurally similar, non-chlorinated coumarin dyes, or to biopolymers conjugated to multiple structurally similar, chlorinated coumarin dyes. The fluorescent biopolymers of the invention typically exhibit absorbance maxima close to 405 nm and are excitable by the principal emission lines of commercially available violet lasers. The fluorescent bioplymers of the present invention, with their unique combination of properties, are particularly useful in biological assays and can provide improved sensitivity in a broad range of assay.

The biopolymer component is an amino acid polymers, which herein is intended to include peptides, proteins, glycoproteins, or polysaccharides, as described further, below. In preferred embodiments, the biopolymer is a monoclonal antibody.

The fluorescent biopolymers of the present invention have the structure of Formula 1:

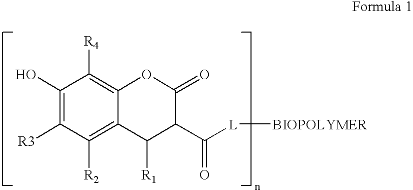

wherein BIOPOLYMER is an amino acid polymer; n is an integer from 3 to 24, more preferably, from 6 to 15; L is an optional linker; either R₃ is chloro and R₄ is H, or R₃ is H and R₄ is chloro; and R₁ and R₂ are independently H, halogen, alkoxy, thiol, alkylthiol, azido, amino, hydroxy, sulfonyl, boronic acid, or alkyl, or alkoxy that is itself optionally substituted one or more times by halogen, amino, hydroxy, sulfonyl, carbonyl or boronic acid.

The dye molecules can be conjugated either directly to the biopolymer or, alternatively, indirectly to the biopolymer through linker, L. For economy of notation, both alternatives are described herein by a single structural formula having an optional linker. In some embodiments, the linker, L, may be an amino acid, a sulfo amino acid, a polyethyleneglycol, or a polyamine.

In a preferred embodiment, the fluorescent biopolymer has a structure of Formula 1, wherein the biopolymer is a monoclonal antibody, and, more preferably, further wherein R₃ is chloro.

In a preferred embodiment, a fluorescent biopolymer of the present invention has the structure of Formula 2:

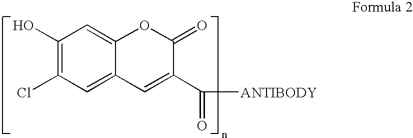

wherein ANTIBODY is a monoclonal antibody; and n is an integer from 3 to 24, more preferably, from 6 to 15.

The antibody preferably is an analyte-specific reagent. Examples of analyte-specific antibodies include antibodies specific for a cell-surface proteins, such as CD3, CD4, CD8, CD11c, CD25, and CD45 antibodies, and antibodies specific for secreted signaling proteins, such as cytokines.

In another aspect, the present invention provides methods of detecting or analyzing an analyte in a sample using a fluorescent biopolymer of the present invention. The fluorescent biopolymer is used as a detection reagent to fluorescently label the analyte through either direct or indirect binding between the biopolymer and the analyte. The fluorescent biopolymers can be used in any of the known assay formats that use fluorescently labeled analyte-specific reagents to label target analytes. Typically, the fluorescent biopolymer and a sample containing the analyte are combined under conditions under which the biopolymer will form a complex with the analyte, and the resulting complexes are detected optically from the fluorescence of the biopolymer. Fluorescence detection of an analyte labeled with a fluorescent biopolymer of the present invention can be carried out using any of the well known methods and instruments. In a preferred embodiment, the fluorescent biopolymers of the present invention are used as detection reagents in either imaging or flow cytometry.

For direct binding to the analyte, the biopolymer can be a member of a specific binding pair, wherein the analyte to be detected is, or has been modified to be, the other member of the specific binding pair. Preferred polymers useful as a member of a specific binding pair are antibodies. Alternatively, for indirect binding to the analyte, the fluorescent biopolymer is, itself, bound to one member of the specific binding pair. The fluorescent biopolymer can be bound either covalently to the binding moiety, or non-covalently, such as through an avidin- or streptavidin-biotin linkage.

In another aspect, the present invention provides kits containing the fluorescent biopolymers of the present invention. Kits of the present invention can contain additional components useful for carrying out the intended application, such as other reagents or buffers.

### DESCRIPTION OF DRAWINGS

FIG. 1 shows the synthesis of reactive coumarins by base-catalyzed condensation of 4-carbonylresorcinols with active methylene compound (Method A) or by acid-catalyzed condensation of resorcinols with beta-oxoacetate compounds (Method B).

FIG. 2 shows the absorption spectrum of a Compound 4-goat anti-rabbit IgG conjugate in pH=9.0 buffer. This conjugate has its maximum absorption around 405 nm.

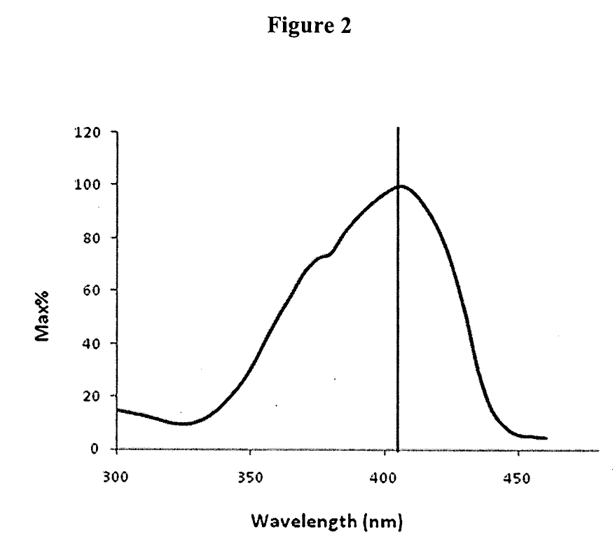

FIG. 3 shows the emission spectrum of a Compound 4-goat anti-rabbit IgG conjugate in pH=9.0 buffer. This conjugate has its maximum emission around 450 nm.

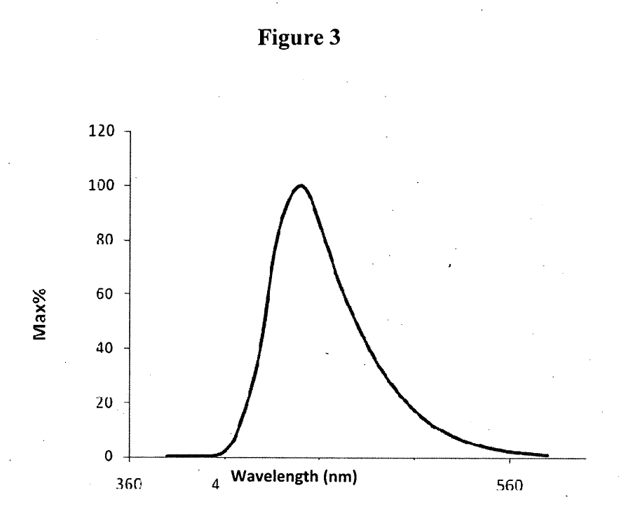

FIG. 4 shows a comparison of the fluorescence brightness of conjugates of Compound 4, Pacific Blue and a non-chlorinated coumarin, CHC (3-carboxy-7-hydroxycoumarin, SE), as described in the examples.

FIG. 5 shows a comparison by flow cytometry of mouse anti human CD45 conjugates prepared from Pacific Blue and Compound 4, as described in the examples.

FIG. 6 shows a comparison by flow cytometry of mouse anti human CD3 conjugates prepared from Pacific Blue and Compound 4, as described in the examples.

FIG. 7 shows a comparison by flow cytometry of mouse anti human CD4 conjugates prepared from Pacific Blue and Compound 4, as described in examples.

### DETAILED DESCRIPTION OF THE INVENTION

In order that the invention herein described may be fully understood, a number of terms are explicitly defined, below. Terms not explicitly defined are intended to have their usual meaning in the fields of chemistry and biology. All references cited herein, both supra and infra, are incorporated herein by reference.

The term “analyte” is used herein broadly to refer to any substance to be analyzed, detected, measured, or labeled. Examples of analytes include, but are not limited to, proteins, peptides, hormones, haptens, antigens, antibodies, receptors, enzymes, nucleic acids, polysaccarides, chemicals, polymers, pathogens, toxins, organic drugs, inorganic drugs, cells, tissues, microorganisms, viruses, bacteria, fungi, algae, parasites, allergens, pollutants, and combinations thereof. By convention, where cells of a given cell type are to be detected, both the cellular component molecules or the cell itself can be described as an analyte.

As used herein an “analyte-specific reagent” or “target-specific reagent” broadly encompasses any reagent that preferentially binds to an analyte or target of interest, relative to other analytes potentially present in a sample. A target (analyte) and target-specific (analyte-specific) reagent are members of a binding pair, and either member of the pair can be used as the target-specific reagent in order to selectively bind to the other member of the pair. Examples of target and target-specific reagent pairs include, but are not limited to, are provided in the Table 1, below. Preferred target-specific reagents are antibodies that include an antigen binding site that specifically binds (immunoreacts with) an antigen.

TABLE 1
Representative specific binding pairs

| Antigen | Antibody |
|---|---|
| Biotin | Avidin, streptavidin, or anti-biotin Antibody |
| IgG (an immunoglobulin) | protein A or protein G |
| Drug | Drug receptor |
| Toxin | Toxin receptor |
| Carbohydrate | Lectin or carbohydrate receptor |
| Peptide | Peptide receptor |
| Nucleotide | Complimentary nucleotide |
| Protein | Protein receptor |
| Enzyme substrate | Enzyme |
| Nucleic acid | Nucleic acid |
| Hormone | Hormone receptor |
| Psoralen | Nucleic acid |
| Target molecule | RNA or DNA aptamer |

As used herein, a “detection reagent” refers to any compound that is used to facilitate optical detection of an analyte. A detection reagent typically comprises an analyte-specific reagent conjugated to a fluorescent label, and includes both fluorescent biopolymers in which the biopolymer component is, itself, an analyte-specific reagent, and analyte specific reagents bound to a fluorescent biopolymer that functions as the fluorescent label.

As used herein, the term “amino acid polymer” is used generically to refer to any polymer of amino acids, including peptides, polypeptides, and proteins, including proteins that have been subject to co-translational or post-translational modification, such as glycoproteins. The amino acid polymer may comprise both standard (i.e., one of the 20 amino acids encoded by the standard genetic code, also referred to as proteinogenic) and nonstandard amino acids, may be derivatized, protected, or substituted, such as, for example, by phosphates, carbohydrates, or C₁ to C₂₅ carboxylic acids. The terms “peptide”, “polypeptide”, and “protein” are used herein interchangeably without a distinction as to the length of the polymer, although short polymers of amino acids are typically referred to as peptides or polypeptides and longer polymers of amino acids, particularly those that are naturally occurring and/or have a biological function, are referred to as proteins.

As used herein, the term “antibody” includes all products, derived or derivable from antibodies or from antibody genes, that are useful as target-specific binding reagents. “Antibody” thus includes, inter alia, natural antibodies, antibody fragments, antibody derivatives, and genetically-engineered antibodies, antibody fragments, and antibody derivatives.

As used herein, a linker between two moieties is referred to as “optional” if the two moieties can be bound either directly to each other or through the linker as an intermediate. This language is used to simplify the description of alternative structures that differ only by the presence or absence of the linker. In the present invention, the coumarin dye molecules can be conjugated either directly to the biopolymer or, alternatively, indirectly to the biopolymer through linker, L. For economy of notation, both alternatives are described herein by a single structure having an optional linker. An embodiment of a structure having an optional linker, L, in which the linker is not present can be described as the structure in which L is “none”.

It is to be understood that the coumarin dyes of the invention have been drawn in one or another particular electronic resonance structure. Every aspect of the instant invention applies equally to dyes that are formally drawn with other permitted resonance structures, as the electronic charge on the subject dyes are delocalized throughout the dye itself.

As used herein, “reactive group (RG)” refers to a moiety on a compound that is capable of chemically reacting with a functional group on a different compound to form a covalent linkage.

The fluorescent biopolymers of the present invention incorporate multiple dyes per biopolymer molecule to increase the fluorescent signal. Preferably, at least 3 molecules of dyes are incorporated into each biopolymer. In embodiments in which the biopolymer is an antibody, at least three, more preferably at least 6 dye molecules are conjugated to the antibody. In some embodiments, as many as about 24 dye molecules can be conjugated to the antibody without significant self-quenching, but more typically as many as about 15. It will be understood by one of skill in the art that each stated range of dye conjugates per biopolymer is intended to describes all values within the range. Thus, for example, by stating that fluorescent biopolymers of the intention contain 6-15 dye molecules, biopolymers containing 6, 7, 8, . . . , or 15 dye molecules are also part of the invention.

Biopolymers

The biopolymer component can be any amino acid polymer, as broadly defined herein. The multiple fluorescent dyes may be bound to any component of the amino acid polymers, such as side groups present on one or more amino acids, or to carbohydrate moieties present on a glycoprotein. Useful biopolymers include both naturally occurring and synthetic molecules and complexes. A number of embodiments of the biopolymer are described in more detail, below.

Examples of amino acid polymers usable in the present invention include, but are not limited to, antibodies (as broadly defined, above), IgG-binding proteins (e.g., protein A, protein G, protein A/G, etc.), enzymes, lectins, glycoproteins, histones, albumins, lipoproteins, avidin, streptavidin, protein A, protein G, phycobiliproteins and other fluorescent proteins, hormones, toxins, chemokines growth factors, neuropeptides, cytokines, toxins, protease substrates, and protein kinase substrates. In a preferred embodiment, the biopolymer is a monoclonal antibody.

Reactive Dyes

The fluorescent biopolymers of the present invention preferably are synthesized as the product of a reaction between the biopolymer and a reactive monochlorinated, 3-carbonyl-7-hydroxycoumarin dye. The reactive dyes of the invention typically have the structure of Formula 3:

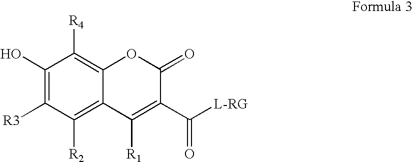

wherein L is an optional linker; either R₃ is chloro and R₄ is H, or R₃ is H and R₄ is chloro; and R₁ and R₂ are independently H, halogen, alkoxy, thiol, alkylthiol, azido, amino, hydroxy, sulfonyl, boronic acid, or alkyl, or alkoxy that is itself optionally substituted one or more times by halogen, amino, hydroxy, sulfonyl, carbonyl or boronic acid.

The reactive mono-chlorinated 3-carbonyl-7-hydroxycoumarins dyes described above can react with a wide variety of biopolymers that contain or are modified to contain functional groups with suitable reactivity, resulting in chemical attachment of dyes to the biopolymer. Typically, the conjugation reaction between the reactive dye and the functional groups on the biopolymer results in one or more atoms of the reactive group RG to be incorporated into a new linkage attaching the dye to the biopolymer.

Typically, a reactive group is an electrophile or nucleophile that can form a covalent linkage through exposure to a corresponding functional group that is a nucleophile or electrophile, respectively. Selected examples of reactive pairs of electrophilic and nucleophilic groups, along with the covalent linkage resulting from their reaction, are shown in Table 2, below.

TABLE 2
Reactive Electrophilic and Nucleophilic groups,
and the Resulting Conjugates.

| Electrophilic Group | Nucleophilic Group | Resulting Conjugate |
|---|---|---|
| activated esters* | amines/anilines | Carboxamides |
| acrylamides | thiols | Thioethers |
| acyl azides** | amines/anilines | Carboxamides |
| acyl halides | amines/anilines | Carboxamides |
| acyl halides | alcohols/phenols | Esters |
| acyl nitriles | alcohols/phenols | Esters |
| acyl nitriles | amines/anilines | Carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic adds | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| imido esters | amines/anilines | amidines |
| isocyanates | amines/anilines | Ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | Esters |
| sulfonate esters | alcohols | Ethers |
| sulfonyl halides | amines/anilines | sulfonamides |
| sulfonyl halides | phenols/alcohols | sulfonate esters |

*Activated esters, as understood in the art, generally have the formula —COW, where W is a good leaving group (e.g., succinimidyloxy (—OC₄H₄O₂) sulfosuccinimidyloxy (—OC₄H₃O₂—SO₃H), -1-oxybenzotriazolyl (—OC₆H₄N₃); or an aryloxy group or aryloxy substituted one or more times by electron withdrawing substituents such as nitro, fluoro, chloro, cyano, or trifluoromethyl, or combinations thereof, used to form activated aryl esters; or a carboxylic acid activated by a carbodiimide to form an anhydride or mixed anhydride —OCOAlk or —OCN(Alk₁)NH(Alk₂), where Alk₁ and Alk₂, which may be the same or different, are C₁-C₂₀ alkyl, C₁-C₂₀ perfluoroalkyl, or C₁-C₂₀ alkoxy; or cyclohexyl, 3-dimethylaminopropyl, or N-morpholinoethyl).
**Acyl azides can also rearrange to isocyanates.

Choice of the reactive group used to attach the dye to the biopolymer typically depends on the functional group on the biopolymer and the type or length of covalent linkage desired. The types of functional groups typically present on the biopolymers include, but are not limited to, amines, amides, thiols, alcohols, phenols, aldehydes, ketones, phosphates, imidazoles, hydrazines, hydroxylamines, disubstituted amines, halides, epoxides, carboxylate esters, sulfonate esters, purines, pyrimidines, carboxylic acids, olefinic bonds, or a combination of these groups. A single type of reactive site may be available on the biopolymer, as is typical for polysaccharides (present in glycolproteins), or a variety of sites may occur (e.g., amines, thiols, alcohols, phenols), as is typical for proteins.

Typically, the reactive group, RG, will react with an amine, a thiol, an alcohol, an aldehyde or a ketone functional group. Preferably, RG reacts with an amine or a thiol functional group. Examples of a reactive group, RG, include an acrylamide, a reactive amine (e.g., a cadaverine or ethylenediamine), an activated ester of a carboxylic acid (e.g., a succinimidyl ester of a carboxylic acid), an acyl azide, an acyl nitrile, an aldehyde, an alkyl halide, an anhydride, an aniline, an aryl halide, an azide, an aziridine, a boronate, a carboxylic acid, a diazoalkane, a haloacetamide, a halotriazine, a hydrazine (including hydrazides), an imido ester, an isocyanate, an isothiocyanate, a maleimide, a phosphoramidite, a reactive platinum complex, a sulfonyl halide, or a thiol group. By “reactive platinum complex” is particularly meant chemically reactive platinum complexes such as described in U.S. Pat. Nos. 5,580,990; 5,714,327; 5,985,566, incorporated herein by reference.

Where RG is an activated ester of a carboxylic acid, the reactive dye is particularly useful for preparing dye-conjugates of proteins. Where RG is a maleimide or haloacetamide, the reactive dye is particularly useful for conjugation to thiol-containing substances. Where RG is a hydrazide, the reactive dye is particularly useful for conjugation to periodate-oxidized carbohydrates and glycoproteins. Preferably, RG is a carboxylic acid, a succinimidyl ester of a carboxylic acid, a haloacetamide, a hydrazine, an isothiocyanate, a maleimide group, an aliphatic amine, a perfluorobenzamido, an azidoperfluorobenzamido group, or a psoralen. More preferably, RG is a succinimidyl ester of a carboxylic acid, a maleimide, an iodoacetamide, or a reactive platinum complex.

Alternatively, the reactive group, RG, is a photoactivatable group, such as an azide, diazirinyl, azidoaryl, or psoralen derivative, in which case the dye becomes chemically reactive only after illumination with light of an appropriate wavelength.

Dye Synthesis

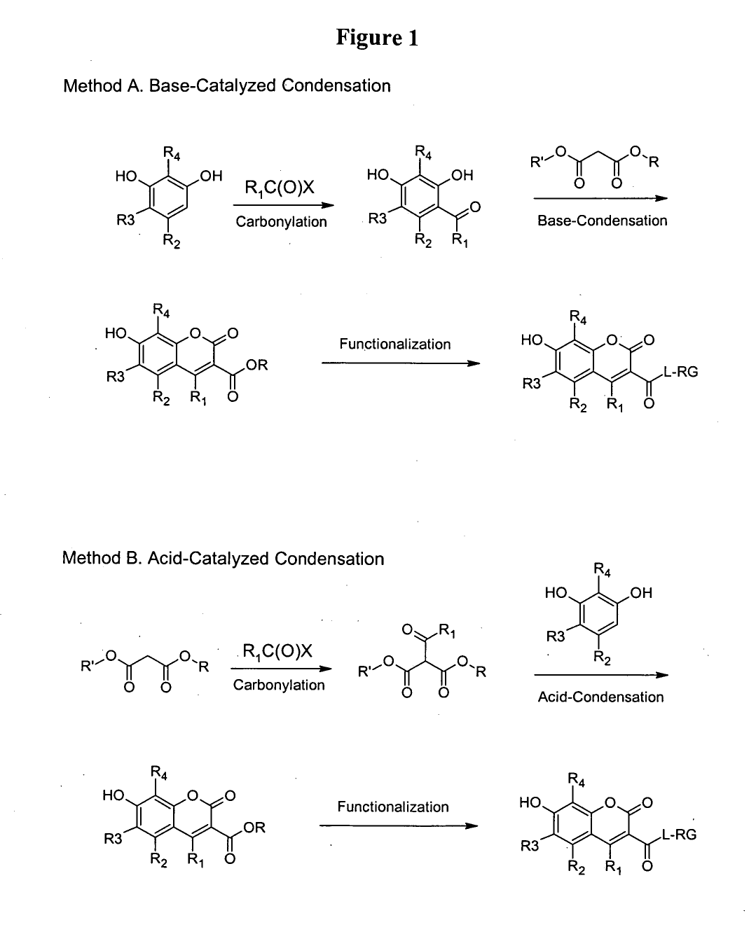

Methods for synthesizing the coumarin dyes of the invention are well known in the literature (Dittmer et al., 2005, J. Org. Chem. 70:4682; Shi et al., 2005, Fen Xi Hua Xue 33:1452; Sivakumar et al., 2004, Org. Lett 6:4603; Zhao et al., 2004, J. Am. Chem. Soc. 126:4653; Huang et al., 1994, J. Chem. Soc. Perkin Trans. 1:102; Kuznetsova and Kaliya, 1992, Russ. Chem. Rev. 61:1243; all of which are incorporated herein by reference). In general, hydroxycoumarins are either prepared from the acid-catalyzed condensation of resorcinols with beta-acylacetate or the base-catalyzed condensation of 4-carbonylresorcinols with active methylene compounds. These basic structures are optionally further substituted, during or after synthesis, to give the corresponding coumarin dye substituents as defined above. The typical total synthesis of different substituted coumarins is illustrated in FIG. 1. It is recognized that there are many possible variations that may yield an equivalent results.

The methods for synthesis of dyes that contain a variety of reactive groups such as those described in Table 2 are well documented in the art. Particularly useful are amine-reactive dyes such as “activated esters” of carboxylic acids, which are typically synthesized by coupling a carboxylic acid to a relatively acidic “leaving group”. Other preferred amine-reactive groups include sulfonyl halides, which are prepared from sulfonic acids using a halogenating agent such as PCl₅ or POCl₃; halotriazines, which are prepared by the reaction of cyanuric halides with amines; and isothiocyanates or isothiocyanates, which are prepared from amines and phosgene or thiophosgene, respectively.

Dyes containing amines and hydrazides are particularly useful for conjugation to carboxylic acids, aldehydes and ketones. Most often these are synthesized by reaction of an activated ester of a carboxylic acid or a sulfonyl halide with a diamine, such as cadaverine, or with a hydrazine. Alternatively, aromatic amines are commonly synthesized by chemical reduction of a nitroaromatic compound. Amines and hydrazines are particularly useful precursors for synthesis of thiol-reactive haloacetamides or maleimides by standard methods.

Preparation of Fluorescent Biopolymers

The fluorescent biopolymers of the present invention typically are synthesized as the product of a reaction between a biopolymer and reactive mono-chlorinated, 3-carbonyl-7-hydroxy-coumarin dye, wherein the reaction conditions result in the conjugation of multiple dye molecules to each biopolymers. Alternatively, the fluorescent biopolymer can be synthesized as a polymerization reaction of subunit molecules, wherein multiple copies of the subunit molecules have been conjugated to a mono-chlorinated, 3-carbonyl-7-hydroxycoumarin dye prior to polymerization of the biopolymer.

The preparation of dye conjugates using reactive dyes is well documented, e.g., Hermanson, 1996, Biocojugate Techniques (Academic Press, New York, N.Y.); Haugland, 1995, Methods Mol. Biol. 45:205-21; and Brinkley, 1992, Bioconjugate Chemistry 3:2, each incorporated herein by reference. Conjugates typically result from mixing appropriate reactive dyes and the substance to be conjugated in a suitable solvent in which both are soluble. Aqueous solutions of the reactive mono-chlorinated, 3-carbonyl-7-hydroxycoumarin dyes described herein are readily created, facilitating conjugation reactions with most biological materials. For those reactive dyes that are photoactivated, conjugation requires illumination of the reaction mixture to activate the reactive dye.

Applications and Methods of Use

In preferred embodiments, the fluorescent biopolymers of the present invention are useful as, or as part of, analyte-specific detection reagents to facilitate the optical detection and analysis of analytes. In one embodiment, the biopolymer itself is an analyte-specific reagent, and the fluorescent biopolymer is used as a detection reagent to label an analyte of interest. In an alternative embodiment, the fluorescent biopolymer is bound to an analyte-specific reagent, and the combined entity is used as detection reagents to label an analyte of interest. In this alternative embodiment, the biopolymer acts as a fluorescent label bound to the analyte-specific reagent.

Assays in which one or more analytes of interest are labeled using analyte-specific detection reagents and subsequently optically analyzed are well known in the art, and the present fluorescent biopolymers are generally useful as detection reagents in such assays. For example, proteins in a sample can be labeled using a detection reagent consisting of a labeled protein, typically an antibody, that binds specifically to the analyte protein. Detection of the resulting labeled analyte proteins can be carried out using a number of well known assay formats and instrumentation, including using flow cytometry, scanning cytometry, imaging, and gel analysis. Flow cytometry is described at length in the extensive literature in this field, including, for example, Landy et al. (eds.), Clinical Flow Cytometry, Annals of the New York Academy of Sciences Volume 677 (1993); Bauer et al. (eds), Clinical Flow Cytometry: Principles and Applications, Williams & Wilkins (1993); Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); and Practical Shapiro, Flow Cytometry, 4th ed., Wiley-Liss (2003); all incorporated herein by reference. Fluorescence imaging microscopy is described in, for example, Pawley (ed), Handbook of Biological Confocal Microscopy, 2nd Edition, Plenum Press (1989), incorporated herein by reference.

For biological applications, the fluorescent biopolymers of the invention are typically used in an aqueous, mostly aqueous, or aqueous-miscible solutions prepared according to methods generally known in the art. The exact concentration of fluorescent biopolymer is dependent upon the experimental conditions and the desired results, but typically ranges from about one nanomolar to one millimolar or more. The optimal concentration is determined routinely by systematic variation until satisfactory results with minimal background fluorescence are accomplished.

Illumination sources useful for exciting the fluorescent polymers of the invention include, but are not limited to, hand-held ultraviolet lamps, mercury arc lamps, xenon lamps, lasers and laser diodes. These illumination sources are optionally integrated into laser scanners, fluorescence microplate readers, standard or mini-fluorometers, or chromatographic detectors. Preferred fluorescent polymers of the invention are excitable at or near 405 nm, and can be excited using a relatively inexpensive violet laser excitation source.

One aspect of the present invention is the formulation of kits that facilitate the practice of various assays using any of the fluorescent biopolymers of the invention, as described above. The kits of the invention typically comprise a fluorescent biopolymer that is a detection reagent. The kit optionally further comprises one or more buffering agents, typically present as an aqueous solution. The kits of the invention optionally further comprise additional detection reagents, luminescence standards, enzymes, enzyme inhibitors, organic solvent, or instructions for carrying out an assay of the invention.

### EXAMPLES

Examples of the synthesis strategies of selected dyes, the synthesis of selected fluorescent biopolymers, their characterization, and methods of use are provided in the examples below. Further modifications and permutations will be obvious to one skilled in the art. The examples below are given so as to illustrate the practice of this invention, and are not intended to limit or define the entire scope of the invention.

### Example 1

### Preparation of Compound 1

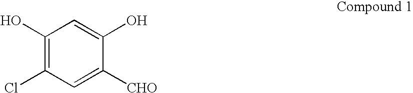

4-Chlororesorcinol (50 g) is dissolved in dry ether (200 ml). To the solution are added finely powdered zinc cyanide (60 g) and potassium chloride (12 g) with stirring. The suspension is cooled to 0° C. A strong stream of hydrogen chloride gas is blown into the solution with vigorous stirring. After approximately 30-60 minutes the reactants are dissolved. The addition of hydrogen chloride gas is continued until it stops being absorbed in the ether solution. The suspension is stirred for one additional hour on ice. The ether solution is poured from the solid that is treated with ice and heated to 100° C. in a water bath. Upon cooling the product crystallized in shiny plates from the solution, which is removed by filtration and air-dried to give the desired aldehyde.

### Example 2

### Preparation of Compound 2

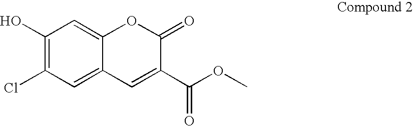

Compound 1 (7 g), dimethylmalonate (6.5 g), 0.5 ml of piperidine and 0.3 ml of acetic acid are heated under reflux for three hours in 100 ml of methanol. After cooling to room temperature, the mixture is filtered and the filtrate is concentrated. The concentrated filtrated is poured into water, and resulted precipitate is filtered off with suction to collect the solid that is air-dried. The crude product is further purified with silica gel chromatography using a gradient of chloroform and ethylacetate as the eluant to yield the desired Compound 2.

### Example 3

### Preparation of Compound 3

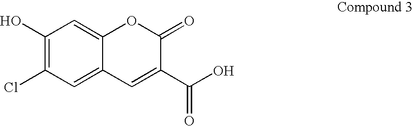

Compound 2 (5 g) is dissolved in methanol (50 ml). To the methanol solution is added 6N HCl (10 ml). The resulted solution is heated at 60-65° C. until Compound 2 is completely consumed. After cooling to room temperature, the mixture is filtered and the filtrate is concentrated. The concentrated filtrated is poured into water, and resulted precipitate is filtered off with suction to collect the solid that is washed with water and air-dried. The crude product is further purified by recrystalization of methanol-water to yield the desired Compound 3.

### Example 4

### Preparation of Compound 4

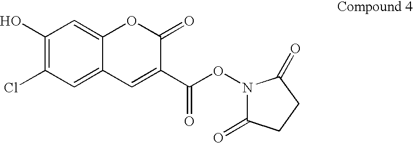

Compound 3 (1 g) and N,N′-disuccinimidyl carbonate (1.6 g) are dissolved in DMF (10 ml). To the DMF solution is added triethylamine (1.2 ml) and 4-dimethylaminopyridine (10 mg). The resulted solution is stirred at room temperature until Compound 3 is completely consumed. The mixture is filtered and the filtrate is concentrated. The concentrated filtrate is poured into water, and resulted precipitate is filtered off with suction to collect the solid that is washed with water and air-dried to yield the desired Compound 4.

### Example 5

### Preparation of Compound 5

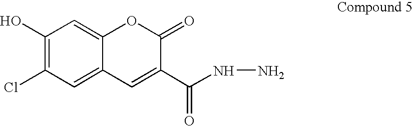

To anhydrous hydrazine (100 μl) in THF (0.5 ml) is added Compound 4 (100 mg) in THF (0.5 ml). The mixture is stirred at ambient temperature for 15 minutes. The reaction solution is poured into water, and resulted precipitate is centrifuged to collect the solid that is washed with water and air-dried. The crude product is further purified by HPLC.

### Example 6

### Preparation of Compound 6

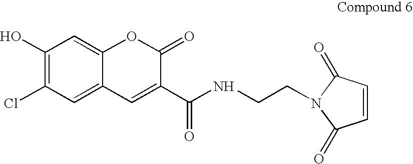

To Compound 4 (100 mg) in DMF (1 ml) at room temperature is added 4 equivalents of triethylamine and 1.2 equivalents of N-(2-aminoethyl)maleimide, trifluoroacetic acid salt (Fanbo Biochemicals, Ltd.). The mixture is stirred at ambient temperature for 60 minutes. The DMF solution is poured into water, and resulted suspension is centrifuged to collect the solid that is air-dried. The crude product is further purified with silica gel chromatography to yield the desired Compound 6.

### Example 7

### Preparation of Compound 7

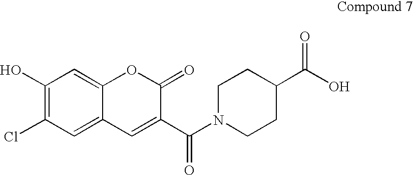

To Compound 4 (100 mg) in DMF (1 ml) at room temperature is added triethylamine (0.2 ml) and isonipecotic acid (40 mg). The mixture is stirred at ambient temperature for 60 minutes. The DMF solution is poured into water, and resulted suspension is centrifuged to collect the solid that is air-dried. The crude product is further purified with silica gel chromatography to yield the desired Compound 7.

### Example 8

### Preparation of Compound 8

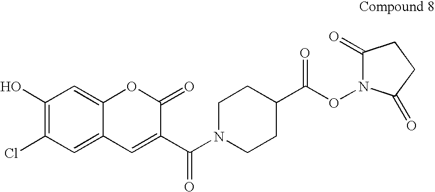

Compound 8 is prepared from the condensation of Compound 7 with N,N′-disuccinimidyl carbonate analogous to the procedure of Compound 4.

### Example 9

### Preparation of Compound 9

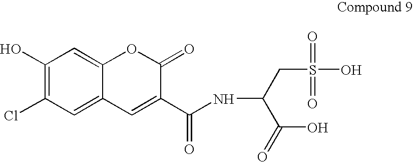

To Compound 4 (100 mg) in DMF (1 ml) at room temperature is added K₂CO₃ (200 mg) and cysteic acid (60 mg). The mixture is stirred at ambient temperature for 60 minutes. The DMF solution is poured into ether, and resulted suspension is centrifuged to collect the solid that is air-dried. The crude product is further purified by HPLC.

### Example 10

### Preparation of Compound 10

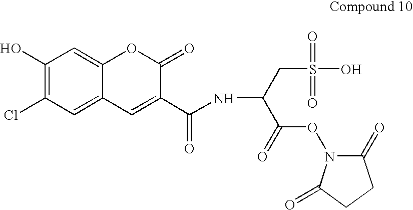

Compound 10 is prepared from the condensation of Compound 9 with N,N′-disuccinimidyl carbonate, analogous to the procedure of Compound 4.

### Example 11

### Preparation of Protein-Dye Coniugates

Conjugates of a protein are prepared by standard means (Haugland et al. 1995, Meth. Mol. Biol. 45:205; Haugland, 1995, Meth. Mol. Biol. 45:223; Haugland, 1995, Meth. Mol. Biol. 45:235; Haugland, 2000, Current Protocols In Cell Biology 16.5.1-16.5.22; each of which is incorporated herein by reference).

A typical method for protein conjugation with succinimidyl esters of the invention is as follows. A solution of the protein is prepared at about 10 mg/mL in 0.1 M sodium bicarbonate. The labeling reagents are dissolved in a suitable solvent such as water, or DMF or DMSO at about 10 mg/mL. Predetermined amounts of the labeling reagents are added to the protein solutions with stirring. A molar ratio of 10 to 50 equivalents of dye to 1 equivalent of protein is typical, though the optimal amount varies with the particular labeling reagent, the protein being labeled and the protein's concentration, and is determined empirically.

Variations in ratios of dye to protein, protein concentration, time, temperature, buffer composition and other variables that are well known in the art are possible that still yield useful conjugates. When optimizing the fluorescence yield and determining the effect of degree of substitution (DOS) on this brightness, it is typical to vary the ratio of reactive dye to protein over a several-fold range. The reaction mixture is incubated at room temperature for one hour or on ice for several hours. The dye-protein conjugate is typically separated from free unreacted reagent by size-exclusion chromatography, such as on Amersham PD-10 resin (GE Healthcare Bio-Sciences Corp., Piscataway, N.J.) equilibrated with phosphate-buffered saline (PBS). The initial, protein-containing colored band is collected and the degree of substitution is determined from the absorbance at the absorbance maximum of each fluorophore, using the extinction coefficient of the free fluorophore. The dye-protein conjugate thus obtained can be subfractionated to yield conjugates with higher, lower or more uniform DOS.

A specific example of preparing a IgG-dye conjugate using Compound 4 is as follows:

Step 1. Preparing protein solution (Solution A): Mix 50 μL of 1 M NaHCO₃ (component B) with 450 μL of IgG protein solution (4 mg/mL) to give 0.5 mL protein sample solution. The resulted solution should have pH 8.5±0.5.

Step 2. Preparing dye solution (Solution B): To 50 μL of DMSO add Compound 4, 8, or 10, and stir until the compound is completely dissolved. The amount of dye added to obtain the desired dye/protein ratio is determined experimentally. Typically, the amount of dye will be in the range of 1-10 mg.

Step 3. Running conjugation reaction: Add the protein solution (A) to the dye solution (B) with effective stirring or shaking, and keep the reaction mixture stirred or shaken for 1-3 hrs.

Step 4. Purifying the conjugate: Dilute 10× elution buffer with de-ionized water to give 1× elution buffer (Solution C) that is used to elute the protein conjugate from PD-10 column. Load the column with the reaction mixture (from step 3, filtrated if necessary) or supernatant as soon as the Iiquid in the pre-packed column runs just below the top surface. Add 1 mL of the 1× elution buffer as soon as the sample runs just below the top resin surface. Repeat this ‘sample washing’ process twice. Add more 1× elution buffer solution to elute the desired sample. Collect the faster-running band that is usually the desired labeled protein. Keep the slower-running band that is usually free or hydrolyzed dye until the desired product is identified.

Step 5. Characterizing the desired dye-protein conjugate: Measure OD (absorbance) at 280 nm and 404 nm (Note: for most spectrophotometers, the sample (from the column fractions) need be diluted with de-ionized water so that the OD values are in the range 0.1 to 0.9). The O.D. (absorbance) 280 nm is the maximum absorption of protein while 404 nm is the maximum absorption of Compound 4 amide (Note: to obtain accurate DOS, the purified conjugate should be free of non-conjugated dye). The DOS is calculated using the following equation:

DOS=[dye]/[protein]=A₄₀₄×εₚ/42000(A₂₈₀−0.19A₄₀₄),

where [dye] is the dye concentration, and [protein] is the target protein concentration. The dye concentration can be readily calculated from the Beer-Lambert Law: A=ε_{dye}C×L, wherein A is the absorbance, ε_{dye} is the molar extinction coefficient, C is the concentration, and L is the length of the light path through the solution. The target protein concentration can be either estimated by the weight (added to the reaction) if the conjugation efficiency is high enough (preferably >70%) or more accurately calculated by the Beer-Lambert Law: A=εₚᵣₒₜₑᵢₙC×L. For example, IgG has the c value to be 203,000 cm⁻¹M⁻¹. For effective labeling, the degree of substitution typically should fall between 6-20 moles of Compound 4 to one mole of antibody.

### Example 12

### Fluorescent Labeling Of Periodate-Oxidized Glycoproteins

Samples of 5 mg of goat IgG antibody (which has a polysaccharide chain attached to the protein) in 1 mL of 0.1 M acetate, 0.135 M NaCl, pH 5.5, are treated with 2.1 mg of sodium metaperiodate on ice for a period of time experimentally determined to be sufficient to result in the desired amount of aldehyde groups on the glycoprotein, which are then reacted with Compound 5. The reactions are stopped by addition of 30 μL ethylene glycol. The antibodies are purified on a Sephadex G25 column packed in PBS pH 7.2. One-tenth volume of 1 M sodium bicarbonate is added to raise the pH and Compound 5 is added at a molar ratio of dye to protein of 50:1. The reaction is stirred at room temperature for a period of time experimentally-determined to be sufficient to result in the desired dye/protein ratio. Sodium cyanoborohydride is added to a final concentration of 10 mM and the reaction is stirred for 4 hours at room temperature. The antibody conjugates are purified by dialysis and on Sephadex G25 columns as described above. Periodate-oxidized glycoproteins in gels and on blots can also be labeled, essentially as described in Estep and Miller, 1986, Anal. Biochem. 157:100-105, incorporated herein by reference.

### Example 13

### Preparation of a Protein-Dye Coniugate Using a Thiol-Reactive Dye

A solution of beta-galactosidase, a protein rich in free thiol groups, is prepared in PBS (2.0 mg in 400 μL). The protein solution is then treated with a 20 mg/L solution of the maleimide derivative Compound 6 in DMF. Unreacted dye is removed on a spin column. The degree of substitution by the dye is estimated using the extinction coefficient of the free dye, as described in Example 11. The protein concentration is estimated from the absorbance at 280 nm, corrected for the absorbance of Compound 6 at that wavelength.

### Example 14

### Comparison of Maximum Fluorescence of Selected Dye-Protein Conjugates

The fluorescence brightness of an IgG-Compound 4 conjugate was compared to IgG-dye conjugates prepared using a non-chlorinated coumarin, CHC (ABD Bioquest, Sunnyvale, Calif.), and Pacific Blue (Molecular Probes, Eugene, Oreg.), the structures of which are shown below. The IgG used in each of the conjugates was a mouse anti-human CD45 antibody (clone 2D1, BD Biosciences, San Jose, Calif.).

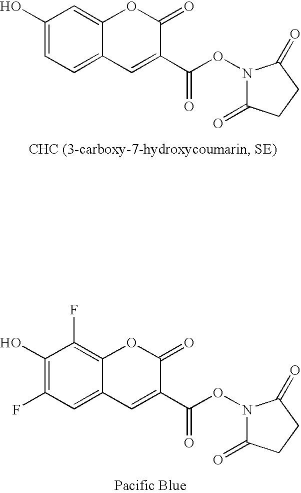

All three conjugates were prepared and characterized under the same conditions, essentially as described in Example 11. The dye/protein ratio for each of the conjugates was as follows:
Compound 4 conjugate, dye/protein=12;
Pacific Blue conjugate, dye/protein=12.4; and
CHC conjugate, dye/protein=11.6.

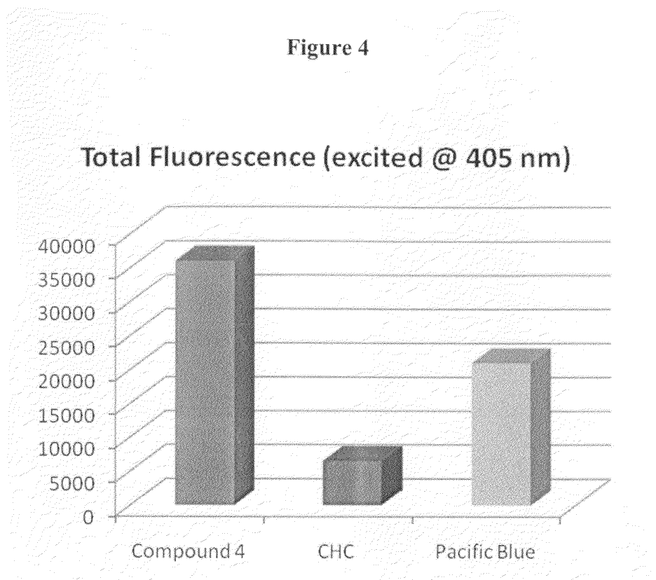

Fluorescence measurements were taken from the conjugates in PBS buffer (pH 7.2) at the same molar concentration. The conjugates were excited at 405 nm and the maximal fluorescence intensities were measured. The results are shown in FIG. 4.

In general, the higher the degree of substitution (DOS), the brighter compounds 4, 10, 14, 16, 18, 20, 22 or 24 bioconjugates are relative to the non-chlorinated coumarin bioconjugates.

### Example 15

### Cell Analysis by Flow Cytometry Using the Fluorescent Biopolymers

Analyte-specific antibodies conjugated to dye compound 4 (fluorescent biopolymers) are used for the analysis of blood cells in, for example, whole blood samples by flow cytometry.

The fluorescent biopolymers are used to label (stain) cellular proteins, and the labeled cells are detected using a flow cytometer.

Samples (100 μL) of whole blood typically are stained with antibody-dye conjugate for 30-60 minutes in the dark. Following staining, 2 mL of 1× FACS™ Lysing Solution (BD Bioscience, San Jose, Calif.) are added to the sample, the sample is mixed at medium speed on vortex mixer, and incubated at room temperature for 10 min. The sample is centrifuged at 300-500 g for 5 min and the supernatant is decanted. The sample is washed (resuspended in 2 mL of a wash buffer, mixed, and centrifuged) twice, re-suspended in either 0.5 mL of a wash buffer or 150 μl of Fixation Stabilization Buffer, and hold at 4° C. until flow cytometric analysis.

Analysis of the stained cells preferably is carried out using a BD LSR II flow cytometer (BD Biosciences, San Jose, Calif.) equipped with a blue (488 nm), a red (˜633 nm), and a violet (405 nm) laser. The detection optics includes detection in a 440/40 nm fluorescence detection channel. Fluorescent biopolymers incorporating dye compound 4 exhibit an excitation maximum closely matching the 405 nm emission of the violet laser, and an emission maximum falling with the 440/40 nm detection channel. The flow cytometer is setup following the manufacturer's instructions. Flow cytometric analysis of the sample of stained cells is carried out according to the manufacturer's protocols, and the data is analyzed using standard techniques well known in the field.

It will be understood that the particular antibody conjugate used and the specific reaction components and particular reaction conditions used can have an effect on the results obtained. Routine experimentation should be carried out to determine preferred reaction components, such as buffers or lyse solutions, and reaction conditions, including staining times and temperatures. Such routine optimization of assay conditions is standard practice in the field of immunostaining-based assays.

### Example 16

### Comparison of Dye/Protein Ratios

Antibody-dye conjugates were prepared using three different antibodies, CD3, CD4, and CD45 antibodies (clones SK7, SK3, and 2D1, respectively, from BD Biosciences, San Jose, Calif.), each conjugated to compound 4 over a range of dye/protein ratios. For comparison, conjugates also were prepared using each of the three antibodies, conjugated to the Pacific Blue (“PB”) over a range of dye/protein ratios. The antibody-dye conjugates were prepared essentially as described in Example 11.

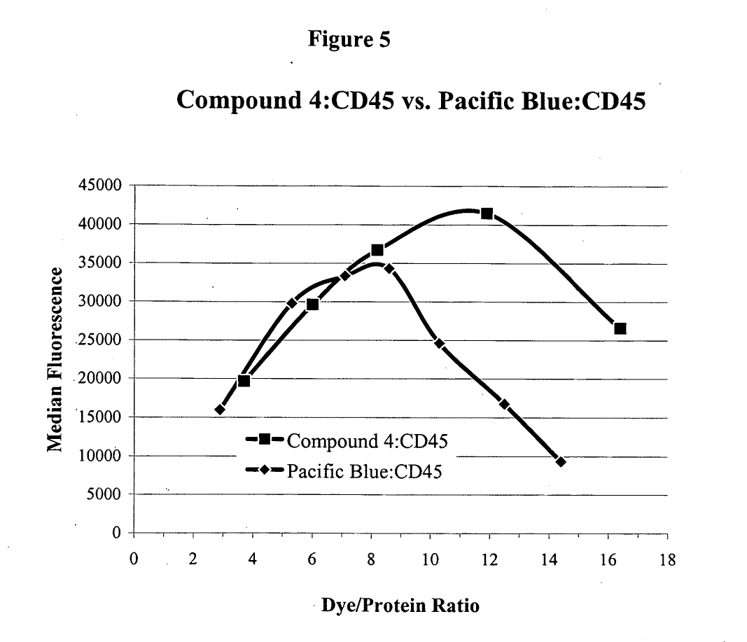

The antibody-dye conjugates were used to analyze whole blood samples by flow cytometry, essentially as described in Example 15. The results are shown in FIGS. 5, 6, and 7.

As seen in FIG. 5, the CD45-Compound 4 conjugates exhibit maximal fluorescence at a dye/protein ratio in range of about 11 to 13. In contrast, the CD45-PB conjugates exhibit maximal fluorescence at a dye/protein ratio in a range of about 7 to 9, and exhibit severe self-quenching at dye/protein ratios around 12. Furthermore, the maximum fluorescence of CD45-Compound 4 conjugates is significantly higher (about 20% higher) than that of the CD45-PB conjugates.

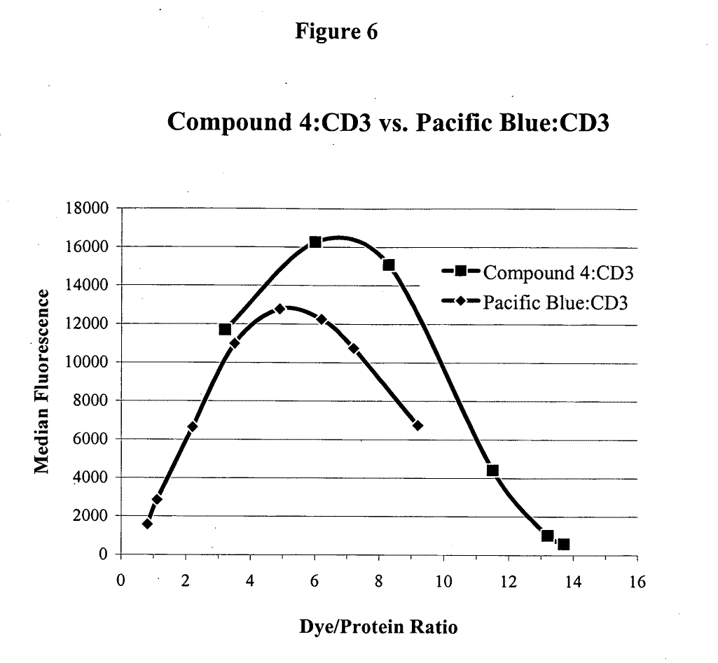

As seen in FIG. 6, the CD3-Compound 4 conjugates exhibit maximal fluorescence at a dye/protein ratio in range of about 6 to 8. In contrast, the CD3-PB conjugates exhibit maximal fluorescence at a dye/protein ratio in a range of about 4 to 6, and exhibit significant self-quenching at dye/protein ratios over 6. Furthermore, the maximum fluorescence of CD3-Compound 4 conjugates is significantly higher (about 30% higher) than that of the CD3-PB conjugates.

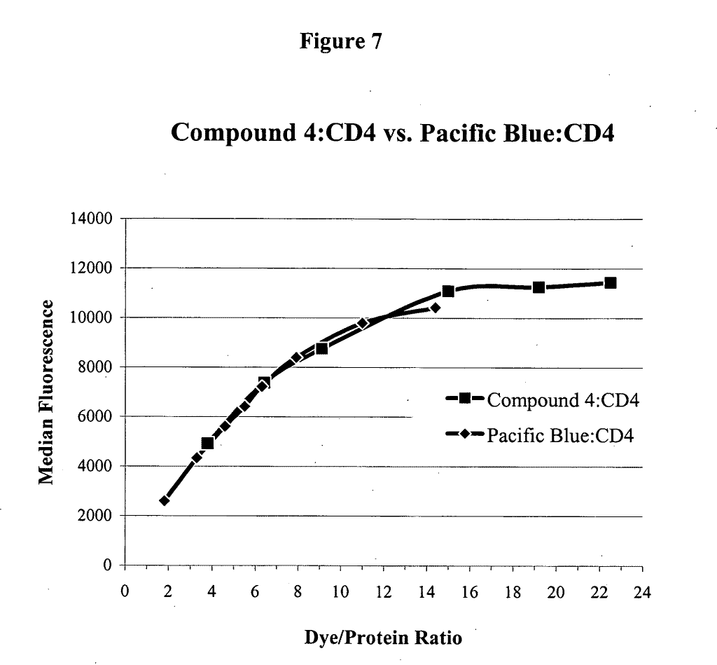

As seen in FIG. 7, the CD4-Compound 4 conjugates exhibit maximal fluorescence at a dye/protein ratio in range of about 15 to 23. Within the range of dye/protein ratios tested, no self-quenching was observed. The CD4-PB conjugates were not tested at dye/protein ratios over about 15, and within this range, no self-quenching was observed. However, based on the fitted curves, the data suggest fluorescence observed from the CD4-PB conjugates at dye/protein ratio of about 15 was the maximum fluorescence obtainable. The data are consistent with the findings that antibody-Compound 4 conjugates can be used at higher dye/protein ratios, and that a higher maximum fluorescence is achieved.

The results show that the mono-chlorinated 7-hydroxycoumarin, Compound 4, can be used to label antibodies at a higher dye/protein ratio than a structurally similar, fluorinated 7-hydroxycoumarin, without resulting in self-quenching, and that a significantly higher maximum fluorescence of the antibody-conjugate can be achieved. The improvement in maximum fluorescence observed was as high as about 30%, although the improvement obtained depended on the particular antibody biopolymer.

### Example 17

### Comparison of Related 7-hydroxycoumarin Dyes

Antibody conjugates of CD45 were prepared using three variants of chlorinated 7-hydroxycoumarin dyes, each conjugated to over a range of dye/protein ratios. The structures of the chlorinated 7-hydroxycoumarin variants are shown below. The antibody-dye conjugates were prepared essentially as described in Example 11.

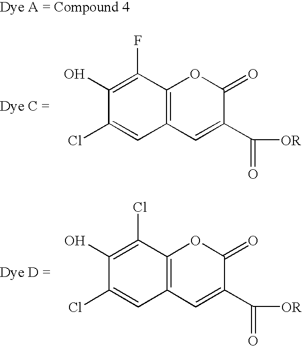

In each, the reactive group, R, was

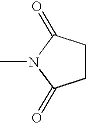

For comparison, a CD45-Pacific Blue conjugate was used, which was conjugated at a dye/protein ratio of 5.6. As can be seen from FIG. 5, this dye/protein ratio is below the optimal dye/protein ratio, but provides a fluorescence emission that is near the optimal emission.

The CD45-dye conjugates (i.e., fluorescent biopolymers) were used in a flow cytometric assay for the analysis of white blood cells. As expected from the data shown in FIG. 5, conjugates using compound 4 exhibited significantly higher maximum fluorescence compared to the conjugate using Pacific Blue, and exhibited a higher optimal higher dye/protein ratios. In contrast, conjugates using either compound C or compound D exhibited worse maximum fluorescence than the conjugate to Pacific Blue, and exhibited optimal dye/protein ratios that were approximately equivalent to the optimal dye/protein ratio for Pacific Blue. Compounds C, D, and Pacific Blue differ from Compound 4 in that both ring positions flanking the 7 hydroxyl group are halogenated, either one chloro and the other fluoro, or both chloro, or both fluoro, respectively. These data show that the unexpectedly advantageous properties of Compound 4 result from a single chloro group flanking the 7 hydroxyl group.

Although not tested, it is expected that conjugates using an 8-chloro, 7-hydroxycoumarin will exhibit essentially the same unexpectedly advantageous properties as exhibited by Compound 4, which is a 6-chloro, 7-hydroxycoumarin. The effect of a single chloro group adjacent to the 7-hydroxyl group is expected to be essentially the same regardless of which side of the hydroxyl group the chloro group is positioned.

It also is expected that minor modifications at ring at positions 4 or 5 (i.e., side group R1 and R2 in structure formula 1) will not have a significant effect on the fluorescent properties of the dye. Such minor modification would include structures wherein R₁ and R₂ are independently H, halogen, alkoxy, thiol, alkylthiol, azido, amino, hydroxy, sulfonyl, boronic acid, or alkyl, or alkoxy that is itself optionally substituted one or more times by halogen, amino, hydroxy, sulfonyl, carbonyl or boronic acid.

### Example 18

### Flow Cytometric Tests of Anti-ERK Conjugates

Anti-ERK-Compound 4 antibody-dye conjugates at several dye/protein ratios were compared to anti-ERK-Pacific Blue conjugates at several dye/protein ratios. The number of ERK-positive cells in a sample of activated peripheral blood mononuclear cells was compared by flow cytometry. In preliminary tests, using this particular anti-ERK antibody and under the particular assay conditions, the unexpectedly advantageous properties observed in other conjugates using Compound 4 were not observed. The reason for this result is not understood, but could be due to the un-optimized reaction conditions. The detection of intracellular proteins, such as ERK, by flow cytometry typically requires different reagents and reaction conditions compared to the detection of cell-surface proteins, as described in the previous examples. Although it cannot be determined without further experimentation, the results obtained with the particular anti-ERK antibody-dye conjugate may be due to inadequate optimization.

What is claimed is:

1. A fluorescent biopolymers having the structure: 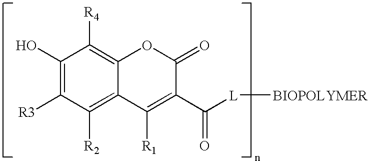
wherein BIOPOLYMER is an amino acid polymer; n is an integer from 3 to 24; L is an optional linker; either R₃ is chloro and R₄ is H, or R₃ is H and R₄ is chloro; and R₁ and R₂ are independently H, halogen, alkoxy, thiol, alkylthiol, azido, amino, hydroxy, su1fonyl, boronic acid, or alkyl, or alkoxy that is itself optionally substituted one or more times by halogen, amino, hydroxy, sulfonyl, carbonyl or boronic acid.

2. The fluorescent biopolymer of claim 1 where amino acid polymer is a monoclonal antibody.

3. The fluorescent biopolymer of claim 1, where n is from 6 to 15.

4. The fluorescent biopolymer of claim 2, where n is from 6 to 15.

5. The fluorescent biopolymer of claim 1 where L is none, an amino acid, a sulfo amino acid, a polyethyleneglycol or a polyamine.

6. A fluorescent biopolymer having the structure: 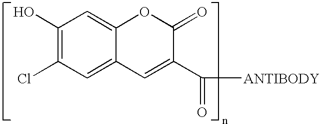
wherein ANTIBODY is a monoclonal antibody; and n is an integer from 3 to 24.

7. The fluorescent biopolymer of claim 6, wherein n is from 6 to 15.

8. The fluorescent biopolymer of claim 6, wherein said antibody is a monoclonal antibody specific for a cell-surface protein or a cytokine.

9. A method of detecting an analyte in a sample, comprising
a) combining said sample with a fluorescent biopolymer having the structure: 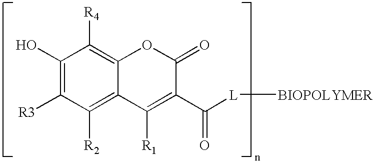
wherein BIOPOLYMER is an amino acid polymer; n is an integer from 3 to 24; L is an optional linker; either R₃ is chloro and R₄ is H, or R₃ is H and R₄ is chloro; and R₁ and R₂ are independently H, halogen, alkoxy, thiol, alkylthiol, azido, amino, hydroxy, sulfonyl, boronic acid, or alkyl, or alkoxy that is itself optionally substituted one or more times by halogen, amino, hydroxy, sulfonyl, carbonyl or boronic acid; wherein said amino acid polymer is, or is bound to, an analyte-specific reagent specific for said analyte, under conditions under which said fluorescent biopolymer will form a complex with said analyte;
b) detecting said complexes.

10. The method of claim 9, wherein said amino acid polymer is a monoclonal antibody.

11. The method of claim 9, wherein n is from 6 to 15.

12. The method of claim 9, wherein L is none, an amino acid, sulfo amino acid, polyethyleneglycol, or a polyamine.

13. A method of detecting an analyte in a sample, comprising
a) combining said sample with a fluorescent biopolymer having the structure: 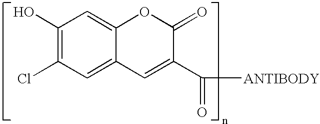
wherein ANTIBODY is a monoclonal antibody that is an antigen-specific reagent for said analyte; and n is an integer from 3 to 24; under conditions under which said fluorescent biopolymer will form a complex with said analyte;
b) detecting said complexes.

14. The method of claim 13, wherein n is from 6 to 15.

15. The method of claim 13, wherein in step b), said complexes are detected by flow cytometry.

16. A kit comprising a fluorescent biopolymer of claim 1.
